(19) United States
(12) Patent Application Publication
(10) Pub. No.: US 2004/0043491 A1
(43) Pub. Date: Mar. 4, 2004

(54) Penicillin production using transgenic merodiploid strains

(76) Inventors: Teresa Suarez Gonzalez, Madrid (ES); Geoffrey Turner, Sheffield (GB); Herbert Arst, Londres (GB); Miguel Angel Penalva Soto, Madrid (ES)

Correspondence Address: KRAMER & AMADO, P.C., 2001 JEFFERSON DAVIS HWY, SUITE 1101, ARLINGTON, VA, 22202, US

(21) Appl. No.: 10/233,584

(22) Filed: Sep. 4, 2002

(51) Int. Cl. C12N 15/74; C12N 1/16

(52) U.S. Cl. 435/484; 435/254.11

(57) ABSTRACT

The object of this invention is a procedure for obtaining a genetically altered merodiploid strain of Penicillium chrysogenum in which the activity of a regulating gene that controls penicillin biosynthesis has been altered, so that this strain is an overproducer of penicillin. This invention represents the first case in which the biosynthesis of a metabolite of industrial interest has been increased by the manipulation of a regulating gene, and therefore is a considerable novelty compared with the previously used technologies.

### FIELD OF THE INVENTION

Biotechnology. Genetic engineering of micro-organisms. Synthesis of antibiotics. Penicillin.

### BACKGROUND OF THE INVENTION

Both Penicillium chrysogenum and the phylogenetically related fungus Aspergillus nidulans synthesise benzylpenicillin (penicillin G) from the same amino acid precursors and phenylacetate. To date, the production of penicillin has been improved by genetic engineering in these fungi by, for example:

(i) An increase in the expression of two or more of the three structural genes responsible for converting the amino acid precursors and phenylacetyl-CoA into penicillin G (Peñalva, M. A. et al. (1998) The optimisation of penicillin biosynthesis in fungi. Trends in Biotechnology 16: 483-489).

(ii) The interruption (in A. Nidulans) of the catabolic route of phenylacetate and its consequent channelling to the biosynthesis of penicillin (Mingot, J. M. et al. (1999) Disruption of phacA, an Aspergillus nidulans gene encoding a novel cytochrome P450 monooxygenase catalysing phenylacetate 2-hydroxylation, results in penicillin overproduction. J. Biol Chem 274: 14545-14550; also Spanish patent P97700833).

(iii) The overexpression (in P. Chrysogenum) of a phenylacetyl-CoA ligase of bacterial origin (Minambres, B. Et al. (1996) Molecular cloning and expression in different microbes of the DNA encoding Psuedomonas putida U phenylacetyl-CoA ligase - Use of this gene to improve the rate of benzylpenicillin biosynthesis in Penicillium chrysogenum. J. Biol Chem 271: 33531-33538; also patent P9600664), and

(i) The channelling of the α-aminoadipic acid precursor to the biosynthesis of penicillin by disruption of the biosynthesis of lysine from said precursor (Casqueiro, J. Et al. (1999) Gene targeting in Penicillium chrysogenum: disruption of the lys2 gene leads to penicillin overproduction. J Bacteriol 181: 1181-1188).

All these strategies are based on the function gain (by overexpression) or the inactivation of structural genes involved in the biosynthesis of penicillin precursors considered individually. This invention covers an alternative possibility that had not been previously explored, which is the manipulation by genetic engineering of a regulating gene to simultaneously increase the function of several structural genes under its control. The advantage is that is can be generally applied to other secondary metabolites and does not require the prior identification of the structural genes the expression of which is altered in the receiving organism.

In A. nidulans (Espeso, E. A: et al. (1993) pH regulation is a major determinant in expression of a fungal penicillin biosynthetic gene. EMBO J. 12:3947-3956) and P. chrysogenum (Suárez, T and Peñalva, ,M. S. (1996) Characterisation of a Penicillium chrysogenum gene encoding a PacC transcription factor and its binding sites in the divergent pcbAB-pcbC promoter of t he penicillin biosynthetic cluster. Mol. Microbiol. 20: 529-540), the penicillin biosynthesis route is positively regulated by the zinc finger protein PacC (Tilburn, J. Et al. (1995) The Aspergillus PacC zinc finger transcription factor mediates regulation of both acid- and alkaline-expressed genes by ambient pH. EMBO J. 14: 779-790). In A. nidulans, PacC is synthesised in an inactive way (674 amino acidic residues) and it is activated by the proteolytic elimination of around 400 amino acids in its carboxy-terminal region. The resulting protein (approximately 250 residues) is a transcription factor of the penicillin biosynthesis genes (Tiburn, J. Et al. (1995) The Aspergillus PacC zinc finger transcription factor mediates regulation of both acid- and alkaline-expressed genes by ambient pH. EMBO J. 14: 779-790; Orejas, M. Et al. (1995) Activation of the Aspergillus PacC transcription factor in response to alkaline ambient pH requires proteolysis of the carboxy-terminal moiety. Gene Develop. 9: 1622-1632). This proteolytic process is produced in response to a signal that is transmitted when the environment is alkaline.

In A. nidulans, mutations (called PacC_{c}) in the pacC gene that produce a truncation in the carboxy-terminal region of the protein between amino acids 263 and 574 (considering the ATG codon in position 5 of the ORF as the main point of transduction initiation) cause the proteolytic activation of the protein, independent of the ambient pH, mimic alkaline ambient conditions and result in a gain in the genic pacC function, so that, whatever the ambient pH, a high expression is produced of the genes that should function with an alkaline pH and a reduced expression of the genes that should function with an acid pH. The “alkaline” genes, the expression of which is increased in this genetic fund, include penicillin biosynthesis route genes.

In A. nidulans, the pacC^{c} mutations behave as codominants in heterozygotic diploids with a wild-type pacC⁺ allele. The behaviour of these mutations in merodiploid strains with two copies of the pacC gene (one of them pacC^{c} and the other pacC⁺) and the rest of the genome in its normal haploid condition is unknown.

In the industrial organism Penicillium chrysogenum there is a homologue of the A. nidulans pacC gene (Suárez, T. And Peñalva, M. A. (1996) Characterisation of a Penicillium chrysogenum gene encoding a PacC transcription factor and its binding sites in the divergent pcbAB-pcbC promoter of the penicillin biosynthetic cluster. Mol. Micriobiol. 20: 529-540). It is possible that this gene (which we will call Pc-pacC) positively regulates the biosynthetic route of penicillin G, in which case an increase in the function could result in an increase in the levels of production of this antibiotic. Pc-pacC (GenBank ID U44726) encodes for a transcription factor (Pc-PacC) that has approximately 64% of amino acid sequence identity with its A. nidulans homologue. The sequence of 641 amino acids deducted for Pc-PacC is shown in SEQ. ID NO 1.

Genetic engineering technology is not very developed in the industrial organism Penicillium chrysogenum in relation to the model organism A. nidulans. It has been shown, for example, that the genic inactivation of a gene by homologous recombination with null alleles is highly inefficient (Peñalva, M. A. et al. (1998) The optimisation of penicillin biosynthesis in fungi. Trends in biotechnology 16: 483-489).

### DESCRIPTION OF THE INVENTION

### Brief Description

The object of this invention is the design by genetic engineering of a genetically altered merodiploid strain of Penicillium chrysogenum, in which the activity of a regulating gene that controls penicillin synthesis has been altered. This invention is the first case in which penicillin biosynthesis has been improved by the manipulation of a regulating gene, and therefore is notably novel in relation to the technologies previously used.

To avoid the previously mentioned technological difficulties, this invention refers to the generation of merodiploid strains that contain, in addition to a wild-type copy of the pacC gene, one or more additional copies of a mutant version of the pacC gene that encodes for a protein truncated at amino acid 477. All these merodiploid strains are notably overproducers of penicillin in relation to the original strain and show high transcription levels of at least two genes of the biosynthesis of the /antibiotic, thus demonstrating the validity of the approach followed.

### Detailed Description of the Invention

A series of merodiploid strains derived from P. chrysogenum NRRL1951. The wild-type PacC gene of this strain encodes a protein of 641 amino acids, the sequence of which is shown on SEQ ID NO 1. In addition to the wild-type pacC gene, this genetically altered strains contain one or several copies of pacC33, an allelic variant of pacC with 481 residues, which encodes for a normal PacC protein up to residue 477, after which, because of a change in the reading pattern that results from truncation, four abnormal amino acids are added, with which it ends at its carboxy-terminal end (see SEQ ID NO 3). The sequence of cDNA nucleotides that this truncated protein encodes is presented in SEQ ID NO 2). The P. chrysogenum pacC gene has an intron starting at nucleotide 223 of 56 pb (not included in SEQ ID NO 2; Suárez, T. and Peñalva, M. A. (1996) Characterisation of a Penicillium chrysogenum gene encoding a PacC transcription factor and its binding sites in the divergent pcbAB-pcbC promoter of the penicillin biosynthetic cluster. Mol. Microbial. 20: 529-540). This truncated protein is designed to provide a gain in the PacC function, through analogy with the A. nidulans situation, regardless of the presence or absence of the signal transduced by the pal gene route.

### Selection of a Receiving P. chrysogenum Strain and Transformation Markers

The transformation marker used to construct several of the genetically altered strains was the sC gene, which encodes for the ATP-sulphurylase enzyme, which converts the sulphate in adenosine 5′-phosphosulphate. This conversion is essential for the use of inorganic sulphate as the only source of sulphur by P. chrysogenum and other fungi, so that sC mutants are incapable of growing in a media with sulphate as a source of sulphur, although they normally do so in a media supplemented with sources of organic sulphate, such as L- or D-methionine. The selection of the transformants in a genetic Sc fund is based on their capacity to grow in a media with sulphate, which distinguishes them from the sC parent strain.

The selenate (SeO₄²-) that penetrates in the cells through the sulphate permease is a toxic compound for fungi. For example, it inhibits the growth of P. chrysogenum NRRL1951 at a concentration of 10 mM, isolating resistant mutants, that can have loss of function mutations in the sB gene (which encodes for the sulphate and selenate permease) or in the sC gene. These two mutant classes are distinguished, for example, because the sB mutants can grow using choline sulphate as the only source of sulphur, whereas the SC mutants do not. Therefore, in the receiving strain, spontaneously selenate-resistant mutants were selected. Once the mutant clones are isolated and purified, we analysed their capacity for growth in different compounds as a source of sulphur to diagnose the inactivated gene in each case by the mutation that created selenate-resistance (H. N. Arst, Jr. (1968) Genetic analysis of the final steps of sulphate metabolism in Aspergillus nidulans. Nature 219: 268-270).

We thus identified several mutants presumably affected in the sC gene. We calculated the reversion frequency of five of these mutants and selected two of them that reverted with a frequency lower than 2×10⁻⁷. The functional sC gene of P. chrysogenum present in the plasmid pINES1 (FIG. 1A) complemented the respective mutations present in these strains, which confirmed that they were both sC mutants. From these, we selected the one with the highest transformation frequency (45 transformations per μg of pINES1). The mutant sC allele from this strain was called sC14. This strain (sC14), which was used as transformation receiver, has been deposited in the CECT with number 20327.

We also used as a transformation marker a chimeric gene in which the phleomycin-resistant bacterial ble gene (antibiotic to which P. chrysogenum is sensitive) is expressed under the control of transcription promoter sequences from the gpda promoter of A. Nidulans and the terminator of the Saccharomyces cerevisiae CYC1 gene. The use of this chimeric gene, which behaves as a dominant marker for the selection of transformants in P. Chrysogenum, has been described by Kolar (Kolar, M. Et al. (1988) Transformation of Penicillium chrysogenum, using dominant selection markers and expression of an Escherichia coli lacZ fusion gene. Gene 62: 127-134). Prior experiments (see Examples) established that a concentration of 1 μg/ml of phleomycin was optimum, for the selection of transformants in the NRRL1951 wild-type strain.

### Preparation of the Transformant Plasmids, Transformation and Selection of the Genetically Altered Strains

A mutant Pc-pacC gene (SEQ ID NO 2) that encodes for a truncated protein with 481 amino acids (SEQ ID NO 3) was introduced in the pPhleo and pPcsC vectors to give rise to the recombinant plasmids pPacC33 (Phleo) and pPacC33 (sC), respectively (FIG. 1B and C). This mutant pc-pacC allele was called pacC33 and carries 1550 pb of the promoter region of the Pc-pacC gene upstream of the ATG transduction initiator. The Pc-pacC gene promoter is a weak promoter (Suárez, T. And Peñalva, M. A. (1996) Characterisation of a Penicillium chrysogenum gene encoding a PacC transcription factor and its binding sites in the divergent pcbAB-pcbC promoter of the penicillin biosynthetic cluster. Mol. Microbiol. 20: 529-540). As a control, we constructed the plasmid pPacC(sC) which is different from pPacC33(sC) in that it has a Pc-pacC⁺ allele instead of pacC33 (see FIG. 1D, plasmid map).

The plasmids we introduced by transformation in P. chrysogenum NRRL1951 (pPacC33(Phleo)) or in its derived mutant strain sC14 (pPacC33 (sC) and pPacC (sC)). After the selection and purification of the transformants and the analysis of the situation and number of copies of the plasmid integrated in the genome by the Southern technique, the following transformants were selected, and deposited in the Spanish Collection of Cultures (CECT): TX5 (CECT 20328) is a strain of P. chrysogenum with 3 copies of pPacC33 (Phleo) integrated in tandem in an undetermined position of the genome. TSC4 (CECT 20329) is a strain of P. chrysogenum with a single copy of pPacC33(sC) integrated in the sC locus. TSC7 (CECT 20330) is a strain of P. chrysogenum with a single copy of pPacC33(sC) integrated in the pacC locus. TSCO3 (CECT 20331) is a strain of P. chrysogenum with a single copy of pPacC(sC) integrated in the pacC locus. All this strains are morphologically indistinguishable from the wild-type strain NRRL1951.

### Results of the Application of this Technology

The merodiploid strains TX5, TSC4 and TSC7 are overproducers of penicillin G, in comparison with the NRRL1951 strain and with its mutant strain NRRL1951 sC14 receiver of the altered genes, with which they did not show differences in growth or in the variance of the pH of the culture media. The production of penicillin and growth rate of the sC14 strain and its parent strain NRRL1951 was very similar, showing that the sC14 mutation does not affect the production of the antibiotic (FIG. 2). Using saccharose as a source of carbon (which normally inhibits production of penicillin G), the merodiploid strains reached, for example, levels at least 5 times greater than those obtained from strain sC14 (see detailed description and FIG. 3). The TSCO3 strain (with two copies of the wild-type pacC gene) is also an overproducer of penicillin compared to strain sC14, although much less than, for example, strain TSC7 with one copy of the wild-type gene and a second copy of the pacC33 allele (FIG. 4). These results validate the principles used in this invention of (i) designing genetically altered strains with the pacC function increased in order to overproduce penicillin and (ii) designing pacC mutations that provide a function gain and behave as dominants in merodiploids with one copy of the wild-type pacC gene.

Strains TX5, TSC4 and TSC7 are also overproducers of penicillin in a lactose media, where the production levels are approximately double those obtained with sC14 or NRRL 1951 (FIG. 5).

The RNA of the different transformants on different days of the penicillin G production was analysed by the Northern technique with specific probes for the structural genes pcbAB, which encodes for the ACV synthetase, and pcbC, which encodes for the IPNS synthetase, from the penicillin biosynthesis route. The quantification with Phosphorimager of the hybridation signals of the membranes, allowed us to determine the transcription profiles of these genes in the receiver strain sC14 cultivated in controlled innoculus and shaking conditions. In this strain, the transcripts of these genes were detected on day 3, with a maximum level on days 5 and 6. For example, the genetically altered strain TSC7 increased approximately twice the times on the maximum transcription level of these two genes in comparison with the sC14 strain (FIG. 6).

To conclude, this invention describes a new procedure that increases the synthesis of penicillin by the genetic manipulation of a regulating gene, pacC. For the first time, a mutant form of this gene is presented in P. chrysogenum, called pacC33, which encodes a protein with a function gain and the sequence of which (SEQ ID NO 2) forms part of this invention.

This information allows us to develop other complete or partial mutant forms, both by synthesis of new DNA sequences and by the isolation and identification of natural forms, of homologue genes in other ascomycetes, which encode new proteins with a function gain in relation to the wild-type protein PacC, and form part of this invention.

The use of promoters for the expression of these mutant forms, different from the promoter of the pacC gene, be they conditional or constitutive promoters, is an evident variant of this invention and forms part of the invention.

These results show that genetically altered merodiploid strains for the pacC gene of P. chrysogenum that have one wild-type allele and another that is an altered pacC mutant, are strains that overproduce penicillin and have high levels of the transcripts of al least two genes involved in penicillin biosynthesis.

The regulating pacC gene controls the synthesis of other secondary metabolites and many extra-cellular enzymes. The use of this technology for the construction of genetically altered strains of P. chrysogenum using the strategy described here (or variants) to positively or negatively alter the pacC function in order to increase the synthesis of useful metabolites or extra-cellular enzymes or to prevent the synthesis of undesirable metabolites such as aflatoxins, is covered by this application.

The transfer of the technology described in this application to other fungi of industrial interest such as, for example, Aspergillus niger or Thricoderma ressei is evident and covered by this application.

The transfer of this technology to other ascomycete-regulating genes in order to increase the synthesis of useful metabolites and extra-cellular enzymes or prevent the synthesis of undesirable metabolites is also covered by this application.

### EXAMPLES

### Example 1

Obtaining Mutant Strains in the Gene that Encodes the ATP-Sulphurylase.

The wild-type strain of P. chrysogenum NRRL1951 was obtained from the CBS (Holland). In order to select selenate-resistant mutants, we prepared 1.5×10⁸ spores of this strain on 30 minimum media dishes (Cove, D. J. (1966) The induction and repression of nitrate reductase in the fungus Aspergillus nidulans. Biochim. Biophys. Act 113: 51-56) with 10 mM of sodium selenate and 10 μg/ml of D-methionine and 1% of glucose and 10 mM of ammonium tartrate, as sources of carbon and nitrogen, respectively. The selenate-resistant mutants appeared with a frequency of 1.5×10⁻⁷ spores. After purification, the phenotype of the mutants was confirmed by growth trials.

The selenate-resistant mutants can map in at least two structural genes, sC (ATP-sulphurylase) and sB (sulphate permease). The mutants in the sC gene do not grow in a media with choline sulphate as a source of S, a compound that is capable of supplementing the deficiency in the permease, since it enters the cell through a permease other than sulphate/selenate. 7 putative sC mutants were thus identified. In order to verify which of them were definitely sC mutants, we proceeded to test by transformation with the plasmid pINES1 (FIG. 1A) if the mutations could be complemented by the functional sC gene of P. chrysogenum, by the selection of the transformants in a media with sulphate as the only source of S. After these experiments, we selected the mutant sC14, since it presented the highest transformation frequency with pINES1 (45 transformants capable of using sulphate/μg of plasmid).

### Example 2

Determination of the Sensitivity of P. Chrysogenum NRRL1951 to Phleomycin.

In order to determine the sensitivity to phleomycin of the NRRL1951 strain, experiments were conducted with protoplasts in regeneration dishes, in the selection of transformant conditions. The protoplasts were obtained after incubating mycelium grown for 20 h at 25° C. in a minimum media (Cove, 1966), with 30 mg of Novozyma per gram of mycelium (drained weight) for 2 h at 25° C. in a 0.9 M KCl, 10 mM phosphate pH 5.8 buffer solution. The protoplasts were briefly shaken in the vortex and centrifuged at 3000 g, so that the protoplasts sedimented on the undigested mycelium, from which they are distinguishable because of their whitish colour. 10⁵ feasible NRRL1951 protoplasts were extended in protoplast regeneration dishes (Cove's minimum media osmotically stabilised with 1M sorbitol and O. M saccharose) that contained 0.05, 0.1, 0.5, 1, 5,10, 20, 40 and 50 μg/ml of phleomycin (Cayla, France). At concentrations higher than 0.5 μg/ml of phleomycin no colony growth was observed after 10 days of incubation at 25°. We therefore routinely used the concentration of 1 μg/ml of phleomycin in the transformation experiments in which a selection based on this antibiotic has been used.

### Example 3

Plasmids Used in the Transformation of NRRL1951.

The plasmid pINES1 (FIG. 1A), from which the sC gene of P. chrysogenum was obtained, is a derivative of pBR322 that includes a 1.5 kb fragment of EcoRI-EcoRV with the pyr4 gene of Neurospora crassa and a 6.1 kb EcoRV-sa/l fragment of the genomic DNA of P. chrysogenum that contains the sC gene. Different plasmids were constructed that carry a wild-type allele or a mutant pacC33 allele of the P. chrysogenum pacC gene. The presence of a Kpnl cutting site in a position of the P. chrysogenum gene similar to where the transduction termination triplet resulting from the pacC_{c}l4 mutation in A. nidulans is found, allowed us to create a protein truncated at residue 477, with 4 additional residues in its carboxy-terminal before reaching a transduction termination codon (SEQ ID NO 2 and 3).

The plasmid pPacC33 (Phleo) (FIG. 1B) was constructed using pBluescript II SK⁺ as a base. This vector was digested with EcoRi and Kpnl and, using conventional genetic engineering techniques, we inserted a 3037 pb EcoRI-Kpnl fragment of genomic DNA of P. chrysogenum NRRL1951, which includes 1553 bp of the Pc-pacC promoter and the encoding region of Pc-pacC up to codon 477 inclusive, to give rise to the pPacC33 plasmid. The resulting mutant allele (called pacC33, SEQ ID NO 2) encodes for the truncated PacC protein described in SEQ ID NO 3. In this plasmid we introduced a chimeric gene consisting on the ble gene of E. coli under the control of promoter signals and fungal terminators, which was obtained from the pHS103 plasmid described by Kolar (Kolar, M. et al. (1988) Transformation of Penicillium chrysogenum using dominant selection markers and expression of an Escherichia coli lacZ fusion gene. Gene 62: 127-134) as a 2.8 kb fragment of EcoRI-HindII, the cohesive ends of which were filled with the DNA polymerase of T4 to proceed to its insertion in the only BamHI site, also converted into blunt by the same method.

The plasmid pPacC33(sC) (FIG. 1C) was constructed in a similar way to pPacC33 (Phleo), except that in this case we inserted in the BamHI site, instead of the phleomycin-resistant gene, an sC function gene, which was obtained from pINES as a 4.3 kb Bg/II fragment (FIG. 1A). The plasmid pPacC(sC) is a derivative of pBS-SK+, in which in the first place we introduced a 7.5 kb EcoRI-Sa/I fragment, which contains the wild-type allele of the pacC gene with the same fragment of the promoter that is present in the pPacC33 (Phleo) and pPacC33(sC) plasmids. Later, the DNA fragment that contains the sC gene was introduced in the same way as in pPacC33(sC).

### Example 4.

Transformation of the NRRL1951 Strain of P. chrysogenum

The transformation of Penicillium was conducted using the protocol described for A. nidulans (Tiburn, J. Et al. (1983) Transformation by integration in Aspergillus Nidulans. Gene 26: 205-211) with slight modifications. The protoplasts were obtained as described in example 2. The protoplasts were re-suspended in STC (sorbitol 1M, 10 mM Tris HCl pH 7.5, 10 mM CaCl₂), they were washed in the buffer twice and they were re-suspended at a concentration of 1-2×107 protoplasts in 200 μl of STC. To each equal part we added 5 μg of circular plasmid (in a volume of less than 20 μl) and 20 μl of PEG 6000 50% (v/v) in STC, and the mixes were incubated for 20 min in ice. Then 1 ml of PEG was added to each tube and they were incubated for 5 min at 25°, 1 ml of STC was added and gently mixed, and they were centrifuged for 5 min at 12000 rpm. The protoplasts were gently re-suspended in 500 μl of STC and then sedimented by centrifugation. Finally, they were re-suspended in 200 μl of STC and extended on osmotically stabilised media dishes (Cove's minimum media (Cove, D. J. (1966) The induction and repression of nitrate reductase in the fungus Aspergillus nidulans. Biochen. Biophys. Act 113: 51-56) with 0.1 M saccharose and 1 M sorbitol), after mixing with 3 ml of the same media with a 0.25% (w/v) of agar. For the selection based on resistance to phleomycin, the antibiotic was included in the regeneration media at a concentration of 1 μg/ml. For the selection based on sC, normal minimum media was used, which contains sulphate as the only source of sulphur. The dishes were incubated at 25° C. The colonies capable of growing in the selective media appeared after 6-7 days, and were purified in a non-stabilised selective media by the isolation of individual colonies that had grown from conidiospores.

### Example 5

Molecular Characterisation of the Transformants

The purified transformants were grown to obtain mycelium, from which the DNA was extracted by the Perez-Esteban method (Perez Esteban, B. Et al. (1993) Molecular characterisation of a fungal secondary metabolism promoter: transcription of the Aspergillus nidulans isopenicillin N synthetase gene is modulated by upstream negative elements. Mol. Microbiol. 9: 881-895). This DNA was digested with the EcoRI or Xbal enzymes to determine the number of copies of the plasmid and its integration site in the genome. The digested DNAs were charged in 0.7% agarose gels to separate the restriction fragments, which were transferred to a nitro-cellulose membrane. This membrane was incubated for 2 h at 42°, in 50% formamide, 5× Denhart solution, 5× SSC and 0.1% SDS with 50 μg/ml of sonicated salmon sperm monocatenary DNA, after which 50 ng of the corresponding probe were added: either the 2.8 kb EcoRI-HindIII fragment that contains the ble gene, or the 4.3 kb Bg/II fragment that contains the sC gene, or a 2.3 kb HindIII-KpnI fragment of P. chrysogenum genomic DNA belonging to the pacC gene, in all cases radioactively marked. The hybridisation was carried out for 18 h at 42°. The final washing of the filters was for 15 min at 65° C. in 0.2× SSC, 0.1% SDS. The filters were exposed to self-radiographic film or Phosphorimager for radioactivity detection.

For the analysis of the transformants, digestions with Xbal were used (FIGS. 7 and 8). The pacC gene probe revealed a 4 kb Xbal band in the sC14 wild-type strain that is transformed into two new 6 and 8.3 kb bands in the TSC7 transformant (in which the plasmid PacC33 (sC) is integrated in the pacC locus, FIGS. 7A and 8A). In the sC14 wild-type strain, the sC gene probe revealed a 7 kb band that is transformed into two 6.5 and 10.8 kb bands in the TSC4 transformant (pPacC33(sC) integrated in locus sC, FIGS. 7B and 8B). The TX5 transformant, obtained with the pPacC33 (Phleo) plasmid, presents, with the pacC gene probe, a 4 kb band, another 10-11 kb band and another close to 9 kb band (FIGS. 7C and 8C). This last band is three times more intense that the band from the resident pacC gene and its mobility corresponds to the size of the plasmid, so it was considered that the TX5 merodiploid is a transformant with 3 tandem-integrated copies in an undetermined position of the genome (FIG. 8C). In a similar analysis, the TSCO3 transformant (FIGS. 7D and 8D) presents, with the pacC probe, the same 4 kb Xbal band that appears in the sC14 strain and a new 8.3 kb fragment (an internal fragment of the pacC insert of ^{˜}2.2 kb and the vector sequences are not detected in this hybridisation. See FIG. 8D).

### Example 6

Production of Penicillin in Saccharose as a Source of Carbon

The selected transformants, together with the control strains of NRRL 1951 or its derivative sC14, were grown at 25° C. with heavy shaking in a penicillin production media (Cove's media supplemented with 2.5% (w/v) of corn steep solids and 0.12% (w/v) of sodium phenylacetate, and 3% of saccharose or 3% of lactose (both w/v) as the main source of carbon). In all cases inoculation was from a suspension of conidiospores, at an initial concentration of 1-2×10⁶ spores/ml, using 500 ml flasks with 100 ml of media. Media samples were taken at different times after inoculation, in which the amount of penicillin produced was measured using a biotest with Serratia marcescens. 1 cm diameter matrices were excavated in solid Antibiotic-l Medium (DIFCO), which included a diluted suspension of the bacteria (at an O.D⁶⁰⁰ of 0.0075). 100 μl of an appropriate supernatant solution was introduced into these matrices. The dishes were incubated for 20 h at 37°, after which the bacterial growth inhibition halo was measured and the amount of penicillin was calculated by comparison with the halos produced by different dilutions of a standard sodium penicillin G solution.

FIG. 2 shows that the level of penicillin production of the sC14 strain is very similar to that of the NRRL 1951 strain from which it is derived, indicating that the mutation does not affect production of the antibiotic. In a medium with saccharose or lactose as the main source of carbon, the growth of the different strains was very similar as far as the biomass measurement and the evolution of the extracellular pH was concerned. However, the genetically altered TX5, TSC4 and TSC7 strains produced significantly higher levels of penicillin than the sC14 parent strain, both with saccharose (FIG. 3) and with lactose (FIG. 5). In the first case, the increase in production was 4.5 times approximately, whereas in lactose, the production levels were approximately twice those obtained with sC14 or NRRL1951 (FIG. 5). The TSCO3 merodiploid strain (with two copies of the pacC wild-type gene) has a growth that is similar to sC14 (see the evolution of the extracellular pH in FIG. 4A) and it is also an overproducer of penicillin compared to the sC14 strain (FIG. 4B), although to a much lesser extent that the TX5, TSC4 and TSC7 strains, which have, in addition to a copy of the wild-type gene, one or more copies of the pacC33 allele (see FIG. 4B for a comparison of the levels of penicillin production of TSC03 with those corresponding to TSC7 in saccharose).

### Example 7

Transcriptional Analysis of the Merodiploids

Mycelium samples were taken from penicillin growth cultures over time (from day 2 to day 10). We extracted the RNA from these mycelium samples using the Lockington method (Lockington, R. A. et al. (1985) Cloning and characterisation of the ethanol utilisation regulon in Aspergillus nidulans. Gene 33: 137-149) and 10 μg of each sample were loaded on 1.2% agarose gels with 18% of formaldehyde in MOPS buffer (40 mM MOPS pH 7.2; 10 mM sodium acetate; 0.4 mM EDTA). The RNAs were transferred to nitro-cellulose membranes which were dried at 80° C. to fix the RNA. These membranes were hybridised with probes that allowed us to detect the following genes: pacC (internal 1.3 kb HindIII-KpnI fragment); pcbC (internal 0.9 kb NcoL-BamHI fragment); pcbAB (2.4 kb EcoRi fragment) and the 955 bp NcoI-BamHI fragment of the acna gene of A. nidulans which encodes for actin, and which was used to control the homogeneousness of the load between the different samples. The hybridisation conditions were identical to those described in example 5, except that the amount of probe added was 100 ng. The final wash was in the same buffer, but at 42° C. In all the Northern experiments a lane with 10 μg of mRNA obtained from a mycelium of the sC14 strain grown for 39 h in a penicillin production medium with 3% of lactose was included in the gel. All the nitro-cellulose membranes, after washing, were exposed for 15-18 h to a Phosphorimager screen sensitive to β emission of the p³² These screens were later read on a Phosphorimager (Molecular Dynamics) and the hybridisation signals were quantified using the ImageQuant software from the same company. In addition to hybridising the membranes with probes that reveal the problem transcripts, all the membranes were hybridised with a probe that reveals the mRNA of actin. The quantifications of the bands revealed with the pcbC, pcbAB and pacC probes were normalised with the intensity obtained on the actin band in each lane. All these quotients for a given gene of a single membrane were normalised to the common lane value (sample of the RNA of the mycelium of the sC14 strain grown in lactose for 39 h(, in order to compare the intensity measurements of different membranes. These measurements, with their standard deviation, appear in FIG. 6, for the pcbAB and pcbC messenger RNAs.

### FIGURES

FIG. 1—Restriction maps of the plasmids employed in the construction of the merodiploid strains of P. chrysogenum. A) pINES plasmid; B) pPacC33 (phleo) plasmid; C) pPacC33 8sC) plasmid; and D) pPacC(sC) plasmid. The different genes are indicated as follows: black, N. crassa pyr4 gene; empty box, Sc gene region of P. chrysogenum (the arrow indicates the approximate position of the gene and the transcription direction); striped box, phleomycin-resistant gene, with the E. coli ble gene in vertical stripes and the transcription promoter and terminator in slanted stripes; finally, the grey box indicates the region of the P. chrysogenum pacC gene, with the position of the wild-type allele and the mutant indicated with an arrow.

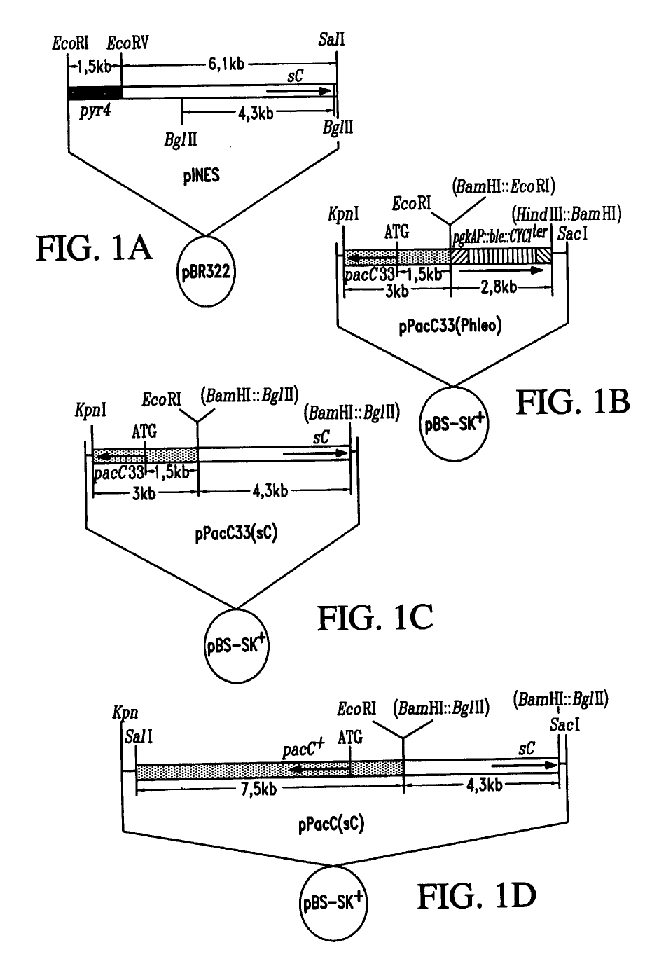

FIG. 2—Growth and production of penicillin from P. chrysogenum NRRL1951 and sC14 strains. The production of penicillin, pH and dry weight in NRRL 1951 and sC14 cultures in a penicillin production medium with 3% of saccharose (A) or lactose (B) as the main source of carbon.

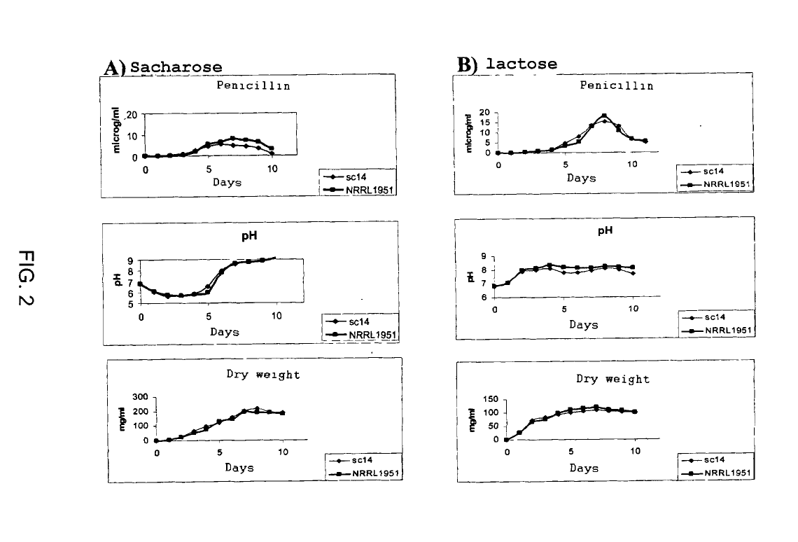

FIG. 3—Growth and production of penicillin from the pacC⁺/pacC33 genetically altered strains. The production of penicillin and the growth rate (pH and dry weight) in cultures of the genetically altered TX5, TSC4 and TSC7 strains, pacC⁺/pacC33 merodiploids, in comparison with the sC14 parent strain. The cultures were in a penicillin production medium with saccharose as the main source of carbon.

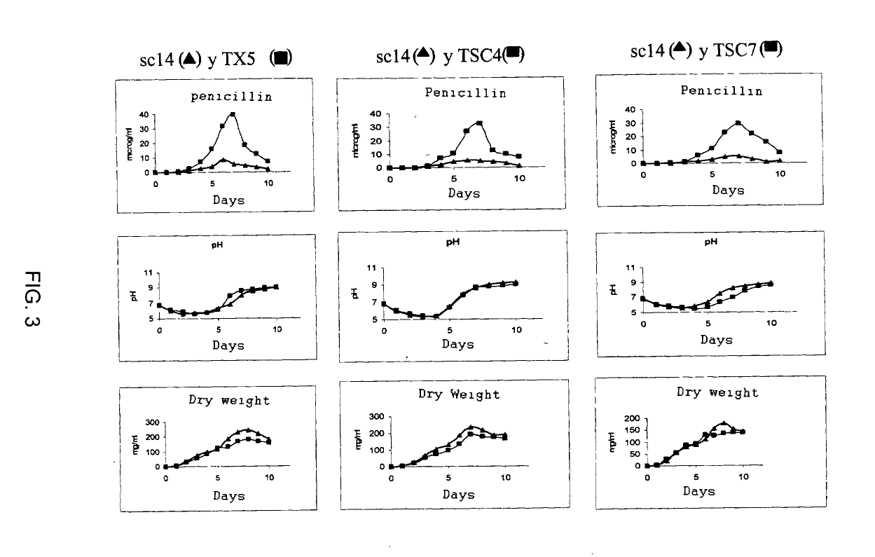

FIG. 4—Growth and production of penicillin from the genetically altered pacC⁺/pacC⁺ strain. The rate of growth (extracellular pH, A) and the production of penicillin (B=from the genetically altered TSCO3 strain, pacC⁺/pacC⁺ merodiploid, in comparison with the sC14 parent strain. The cultures were in a penicillin production medium with saccharose as the main source of carbon. In panel B, we also observe the difference in the production of penicillin between the pacC⁺/pacC⁺ merodiploid (TSC03) and a pacC⁺/pacC33 merodiploid (TSC7)

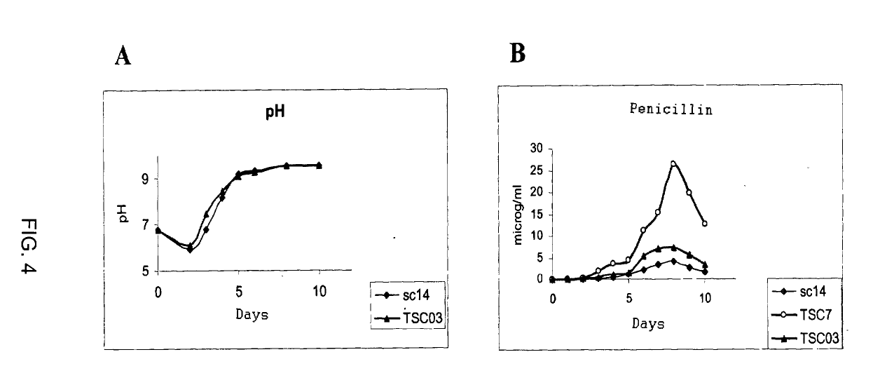

FIG. 5—Production of penicillin in a media with lactose. The production of penicillin in lactose from the pacC⁺/pacC33 merodiploid strains TSC4, TSC7 and TX5, in comparison with the sC14 parent strain.

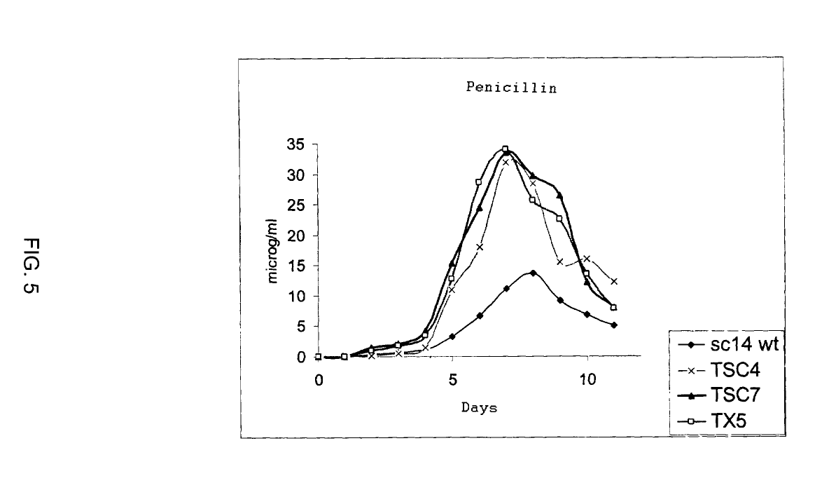

FIG. 6—Quantification of the pcbC and pcbAB mRNA levels in the sc14 and TSC7 strains. The measurements are expresses in arbitrary units (AU). Culture time is indicated on abscissas. The data is the average of three experiments and the error bars indicate the standard deviation.

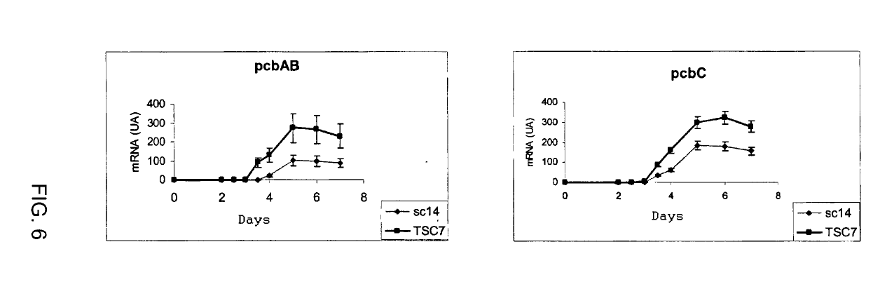

FIG. 7—Southern method analysis of the merodiploid strains of P. chrysogenum. The DNA samples from the different strains were digested with XbaI. The probe used was a fragment of the pacC gene (A, C and D) or the sC gene (B). The arrows indicate the hybridisation bands obtained with the merodiploids and the receiver strain, as indicated in the text.

FIG. 8—Graphic representation of plasmid recombination. The graphic interpretation of the bands revealed in the Southern on

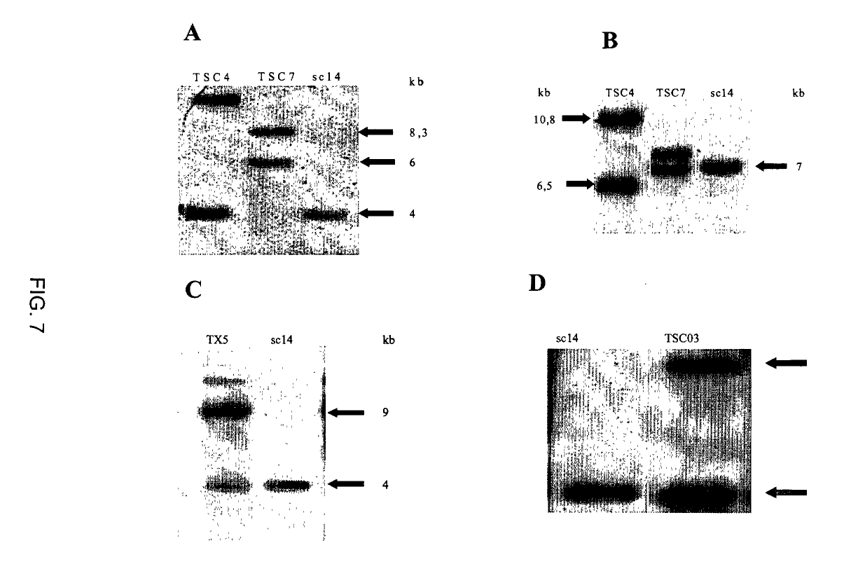

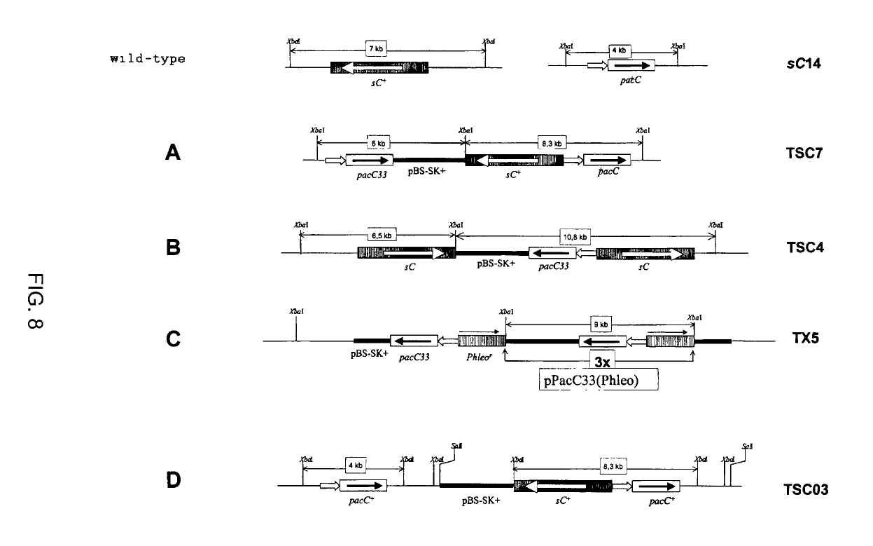

FIG. 7 is represented here for the sc14 (wild-type), TSC7 (A), TSC4 (B), TX5 (C) and TSCO3 (D) strains. The genome of the fungus is indicated by the fine continuous line. The box with thick stripes represents the sC gene (wild or mutant versions, sc14) and the white arrow indicated the transcription direction. The pacC⁺ or pacC^{c}33 mutants are represented by a white box with an internal arrow (which indicates the transcription direction). The phleomycin-resistant gene is indicated by a box with thin stripes and the plasmidic sequences by a continuous thick line. It is not possible to determine the integration site for the TX5 transformant, and we indicate the repetition of three copies of the transformant plasmid. The measurement lines show the sizes (in kb) of the fragments indicated in the hybridisation on FIG. 7.

1. A method for obtaining genetically altered strains of filamentous fungi characterised in that:
a) the genetically altered strains are merodiploids for a regulating gene,
b) the method is based on the transformation of these strains with an integrating plasmid that includes a sequence of nucleotides that corresponds to a dominant or co-dominant wild-type or mutant allele of the this regulating gene, and in that
c) these strains present new function-gain or -loss capacities in the production of metabolites or extracellular enzymes.

2. The method according to claim 1, characterised in that there is a function gain of the regulating gene.

3. The method according to claim 1, characterised in that there is a function loss of the regulating gene.

4. The method according to any of claims 1 to 3, characterised in that the production of a secondary metabolite is increased.

5. The method according to any of claims 1 to 4, characterised in that the secondary metabolite is a penicillin or any other beta-lactamic antibiotic produced by filamentous fungi.

6. The method according to any of claims 1 to 5, characterised in that the sequence of nucleotides can be a genomic or DNAc version of the regulating gene, obtained by synthesis or selection.

7. A genetically altered merodiploid strain obtained by the method according to any of claims 1 to 6, where the filamentous fungus belongs to the group of ascomycetes of economic or medical interest, although it is not restricted to these, such as Penicillium chrysogenum, Apersgillus niger, Aspergillus nidulans, Thricoderma ressei and Candida albicans.

8. The sequence of nucleotides according to claim 1, characterised in that the sequence represents an allele of the pacC regulating gene of Penicillium chrysogenum and encodes for a mutant protein with a function gain.

9. The sequence of nucleotides according to claim 1, characterised in that the sequence represents an allele of the pacC regulating gene of Penicillium chrysogenum and encodes for a mutant protein with a function loss.

10. The sequence of nucleotides according to claim 8, characterised in that the sequence consists of SEQ ID NO 2 and it is the pacC33 allele of the pacC regulating gene.

11. The sequence of nucleotides characterised in that the sequence presents an identity of at least 30% with the sequence according to any of claims 8 to 10.

12. The plasmid according to claim 1, characterised in that the plasmid contains a sequence of nucleotides according to any of claims 8 to 11 and all the elements required to promote the expression of the regulating gene that corresponds to that sequence.

13. The plasmid according to claim 12, characterised in that the selective marker is the P. chrysogenum sC gene.

14. The plasmid according to claim 12, characterised in that the selective marker is the sC gene of others Peniciliia, A. nidulans and other Aspergilli and other filamentous or yeast-type ascomycetes.

15. The plasmid according to claim 12, characterised in that the plasmid presents the identifying features of the pPacC33 plasmid.

16. The plasmid according to claim 12, characterised in that the plasmid presents the identifying features of pPacC22(sC).

17. The plasmid according to claim 12, characterised in that the plasmid presents the identifying features of pPacC33(Phleo) of this invention.

18. The plasmid according to claim 12, characterised in that the plasmid includes a sequence of nucleotides that encodes for any function gain or loss allele of the P. chrysogenum pacC gene.

19. The plasmid according to claim 12, characterised in that the plasmid includes the sequence of nucleotides that encodes for any function gain or loss allele of the pacC regulating gene of other Penicillia, other Aspergilli and other filamentous or yeast-type ascomycetes.

20. The plasmid according to claim 1, characterised in that the plasmid contains the sequence of nucleotides of the wild type of the pacC regulating gene of P. chrysogenum.

21. The plasmid according to claim 20, characterised by the identifying features of the pPacC(sC) plasmid.

22. The plasmid according to claim 1, characterised in that the plasmid contains the sequence of nucleotides of the wild type of the pacC regulating gene of other Penicillia, other Aspergilli and other filamentous or yeast-type ascomycetes.

23. The genetically altered merodiploid strain according to claim 7, characterised in that the strain contains a plasmid that includes a wild-type or mutant allele of the pacC regulating gene.

24. The genetically altered merodiploid strain according to claim 23, characterised in that the mutant allele of the pacC gene is a function-gain allele.

25. The genetically altered merodiploid strain according to claim 23, characterised in that the mutant allele of the pacC gene is a function-loss allele.

26. The genetically altered merodiploid strain according to any of claims 7 and 23 to 25, characterised in that it contains a plasmid according to any of claims 12 to 22.

27. The genetically altered merodiploid strains according to claim 26, characterised in that they present the identifying features of the P. chrysogenum strains registered in the CECT with numbers 20328, 20329, 20330 and 20331.

28. A method of using the genetically altered merodiploid strains according to claim 7, for the production of metabolites and extracellular proteins of industrial interest.

29. A method of using the genetically altered strains according to any of claims 23 to 27 for the production of penicillin.


SEQUENCE LISTING (3 sequences)

SEQ ID NO: 1 - protein, 641 aa, Penicillium chrysogenum
MTENHTPSTTQPTLPAPVAEAAPIQANPAPSASVTATAATAAVNNAPSMNGAGEQLPCQWVGCTEKSPTAESLYEHVCERHVGRKSTNNLNLTCQWGTCNTTTVKRDHITSHIRVHVPLKPHKCDFCGKAFKRPQDLKKHVKTHADDSEIRSPEPGMKHPDMMFPQNPRGSPAATHYFESPINGINGQYSHAPPPQYYQPHPPPQAPNPHSYGNLYYALSQGQEGGHPYDRKRGYDALNEFFGDLKRRQFDPNSYAAVGQRLLGLQALQLPFLSGPAPEYQQMPAPVAVGGGGGGYGGGAPQPPGYHLPPMSNVRTKNDLINIDQFLEQMQNTIYESDENVAAAGVAQPGAHYVHGGMNHRTTHSPPTHSRQATLLQLPSAPMAAATAHSPSVGTPALTPPSSAQSYTSNRSPISLHSSRVSPPHEEAAPGMYPRLPAAICADSMTAGYPTASGAAPPSTLSGAYDHDDRRRYTGGTLQRARPAERAATEDRMDISQDSKHDGERTPKAMHISASLIDPALSGTSSDPEQESAKRTAATATEVAERDVNVAWVEKVRLLENLRRLVSGLLEAGSLTPEYGVQTSSASPTPGLDAMEGVETASVRAASEQAREEPKSESEGVFYPTLRGVDEDEDGDSKMPE

SEQ ID NO: 2 - DNA, 1446 bp, Penicillium chrysogenum
  CDS (1)..(1446)
atgacggagaaccacaccccttctactacgcagccgacgttgcctgcgcctgttgctgaagccgcgccgatccaagcaaacccggctccttctgcctcagtcacggcgactgctgctactgcggcggtgaacaacgccccctctatgaacggcgccggtgagcagttgccttgccagtgggttggttgcacggagaagtcccccactgccgagtctctatatgagcatgtttgcgagcgtcatgttggacgtaaaagcaccaacaacctcaacctgacctgccagtggggcacttgcaacaccacaacagtcaagcgtgatcatatcacctcccacatccgcgttcatgtgccacttaagccgcacaaatgcgacttttgtggtaaggctttcaagcgcccccaggatttgaagaagcatgtcaagactcatgcggacgactccgagatccgctcccccgaaccgggcatgaagcaccctgatatgatgttcccccaaaaccctaggggttcccctgctgccacacattacttcgaaagccctatcaacggcatcaatgggcaatattcacatgcaccgcctccccagtactaccagccacaccccccaccccaggctcccaacccgcattcctacggcaatctatactatgccctgagccaaggacaagagggaggccacccctacgaccgtaagcgcggatatgacgcgttgaacgaattttttggcgacttgaagcgccgccagttcgaccctaattcctatgccgcggtcggccagcgtctgctgggtctccaggcccttcagcttcccttcctcagtggccctgcccccgaataccagcaaatgcctgcgcctgttgccgttggcggcggcggtggtggttatggcggtggtgctccccagcctcctggttaccacctgccccccatgtccaatgttcggactaagaacgatttgatcaacattgatcagttcctcgaacaaatgcagaacactatctacgagagcgatgagaatgtggctgctgccggtgttgcccagcccggcgcgcattacgtgcacggtggcatgaatcatcgcaccacccactctcccccaacccactcccgccaagccacgttactgcaactaccttcagcccccatggcggctgctacagcgcactccccatcggtcggcaccccagccctgaccccaccttccagcgcacagtcgtatacctccaaccgctctcccatctccctgcacagctcacgcgtgtcgccccctcacgaggaggcggcgccgggtatgtaccctcgcttgcctgcggccatctgcgccgacagcatgactgcaggctatccgaccgcctcaggtgccgcaccaccctctactctgagcggtgcgtatgaccacgatgaccgccgccgctacactggtggtacccaattcgccctatag

SEQ ID NO: 3 - protein, 481 aa, Penicillium chrysogenum
MTENHTPSTTQPTLPAPVAEAAPIQANPAPSASVTATAATAAVNNAPSMNGAGEQLPCQWVGCTEKSPTAESLYEHVCERHVGRKSTNNLNLTCQWGTCNTTTVKRDHITSHIRVHVPLKPHKCDFCGKAFKRPQDLKKHVKTHADDSEIRSPEPGMKHPDMMFPQNPRGSPAATHYFESPINGINGQYSHAPPPQYYQPHPPPQAPNPHSYGNLYYALSQGQEGGHPYDRKRGYDALNEFFGDLKRRQFDPNSYAAVGQRLLGLQALQLPFLSGPAPEYQQMPAPVAVGGGGGGYGGGAPQPPGYHLPPMSNVRTKNDLINIDQFLEQMQNTIYESDENVAAAGVAQPGAHYVHGGMNHRTTHSPPTHSRQATLLQLPSAPMAAATAHSPSVGTPALTPPSSAQSYTSNRSPISLHSSRVSPPHEEAAPGMYPRLPAAICADSMTAGYPTASGAAPPSTLSGAYDHDDRRRYTGGTQFAL